(19) United States
(12) Patent Application Publication
(10) Pub. No.: US 2021/0244292 A1
(43) Pub. Date: Aug. 12, 2021

(54) OPTICAL MEASUREMENT OF VENOUS DISTENSION

(71) Applicant: CardioDriven, Inc., Parkland, FL (US)

(72) Inventor: David F. Snipelisky, Parkland, FL (US)

(21) Appl. No.: 16/784,240

(22) Filed: Feb. 6, 2020

(51) Int. Cl. A61B 5/02; G06N 3/04; G06N 3/08; G01J 5/00; A61B 5/00

(52) U.S. Cl. CPC A61B 5/02007; G06N 3/04; G06N 3/08; A61B 5/0002; A61B 5/0075; A61B 5/743; G01J 5/0025

(57) ABSTRACT

Embodiments of the present invention provide a method, system and computer program product for optically detecting a jugular vein (JV) and measuring distension therein. In an embodiment of the invention, a method for optically detecting an JV includes switching power in an JV probe to an array of light emitters emitting infrared light above seven-hundred nanometers (700 nm) onto a target area of a neck, measuring in the JV probe an intensity of reflected portions of the infrared light, and locating the JV on the neck responsive to the measured intensity falling below a threshold value. In this regard, the infrared light may have a wavelength ranging from seven-hundred forty nanometers (740 nm) to eight-hundred fifty (850 nm).

### BACKGROUND OF THE INVENTION

### Field of the Invention

The present invention relates to the field of reflection-type pulse wave measurement of blood flow.

### Description of the Related Art

As the heart pumps blood through the vascular system, the change in fluid pressure may be observed remotely by way of reflection-type pulse wave velocity measurement. In reflection-type pulse wave measurement, an optical light source emits light waves towards the target blood vessel and an optical light sensor measures the amount of light reflected. To the extent that elements of the blood flowing through the target vessel absorb the emitted light waves, a volume of the blood flowing through the target vessel may be correlated to the amount of the light reflected relative to the amount of light emitted. The foregoing is the foundation of pulse measurement and blood oxygen saturation measurement performed by a wearable device such as a fitness watch or smart watch.

Generally, within the visible spectrum of light beginning at four-hundred nanometers (400 nm) and extending to seven-hundred nanometers (700 nm), red or near infrared light ranging from six-hundred eighty nanometers (680 nm) to the nominal red edge of the visible spectrum at 700 nm is preferred as the medium of measurement for reflection-type pulse wave measurement. However, in an outdoor setting, to the extent that infrared energy from sunlight might affect the measurement of reflected near infrared light, lower wavelength green light is preferred. To wit, hemoglobin is known to have a high absorption rate with respect to green light so as to produce a better contrast for measuring light reflection.

While reflection-type pulse wave measurement has proven to be an effective tool in respect to the measurement of heart rate, blood oxygen saturation measurement and even heart rate variability (stress) at the wrist, applying the same technique using the same light source at other parts of the body with respect to other blood vessels is not so effective. To wit, not all blood vessels and blood flowing therethrough are the same. Some blood vessels support substantially larger volumes of flowing blood at various depths within the body and at various cross-sectional diameters. Further, the content of blood flowing through a target vessel of interest may not demonstrate optimal absorption at the customary wavelengths of sub-700 nm or 400 nm as is the case with traditional reflection-type pulse wave measurement.

In this regard, it is widely recognized that the detection of flow abnormalities in jugular venous (JV), for instance the internal jugular vein (IJV), can be most helpful in diagnosing important cardiac states such as right ventricular failure, tricuspid stenosis, tricuspid regurgitation and cardiac tamponade. The distension of the JV can be indicative of any of the foregoing conditions. At present, a health care provider attempts to discern the distension of the JV visually by placing the head of the subject at a forty-five degree angle and slight turning the head away from the observing health care provider so as to expose a view to the JV and to observe a bulging of the JV in the event of a distension of the JV. The health care provider then measures the height of the bulge in order to estimate the central venous pressure within the JV. The foregoing measurement, though, is performed by the health care practitioner in office. But, given the direct correlation between distension of the IJV and heart failure, however, being able to measure the distension of the JV outside of the office of the health care practitioner would be very desirable.

### BRIEF SUMMARY OF THE INVENTION

Embodiments of the present invention address deficiencies of the art in respect to JV location and JV distension measurement and provide a novel and non-obvious method, system and computer program product for optically detecting a JV and measuring distension therein. In an embodiment of the invention, a method for optically detecting a JV includes switching power in a JV probe to an array of light emitters emitting infrared light above seven-hundred nanometers (700 nm) onto a target area of a neck, measuring in the JV probe an intensity of reflected portions of the infrared light, and locating the JV on the neck responsive to the measured intensity falling below a threshold value. In this regard, the infrared light may have a wavelength ranging from seven-hundred forty nanometers (740 nm) to eight-hundred fifty (850 nm).

In one aspect of the embodiment, the method further includes collecting by the JV probe over a period of time, different values of the measured intensity of the reflected portions of the infrared light at the location of the JV of the neck, and displaying the different values in a time-based graph in a display coupled to the JV probe. In yet another aspect of the embodiment, the method additionally includes correlating the measured intensity at the location for each of the different values with corresponding degrees of distension and displaying the corresponding degrees of distension in the display coupled to the JV probe.

In another embodiment of the invention, JV optical detection system includes a JV probe that has a rigid-flexible printed circuit board substrate, a host computer with a processor, memory, and wireless communications circuitry affixed to a rigid portion of the substrate, and an array of light emitters emitting infrared light above 700 nm and a corresponding array of infrared light sensors both affixed to a flexible portion of the substrate. The system also includes a JV detection module. The module includes computer program instructions that when executing in the memory of the JV probe are enabled to switch power to the array of light emitters onto a target area of a neck, measure an intensity of reflected portions of the infrared light and locate the JV on the neck responsive to the measured intensity falling below a threshold value.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is pictorial illustration of a process for optically detecting a JV;

FIG. 2 is a schematic diagram of computer data processing system adapted for JV optical detection; and,

FIG. 3 is a flow chart illustrating a process for optically detecting a JV.

### DETAILED DESCRIPTION OF THE INVENTION

Embodiments of the invention provide for the optical detection of a JV and the optical measurement of the distension of a located JV. In accordance with an embodiment of the invention, an array of light emitting diodes present in an JV probe and emitting infrared light with a wavelength greater than 700 nm, emits light onto a target area of the neck. A corresponding array of photodiodes in the JV probe receives reflected portions of the light and quantifies and intensity of the reflected portions. Upon a determination that the intensity falls below a threshold value indicative of a peak absorption of the emitted light by the JV, the JV is located on the neck. Thereafter, as light continues to be emitted at the location of the JV, reflected portions are sensed in the photodiodes and an intensity of the reflected portions measured. The measured intensities are then correlated formulaically, by reference to a table, or through submission to a neural network, with a corresponding distension value and presented to the operator of the JV probe. In this way, the location and distension of the located JV can be determined with precision and consistency at any time—not just within the office setting of the health care practitioner.

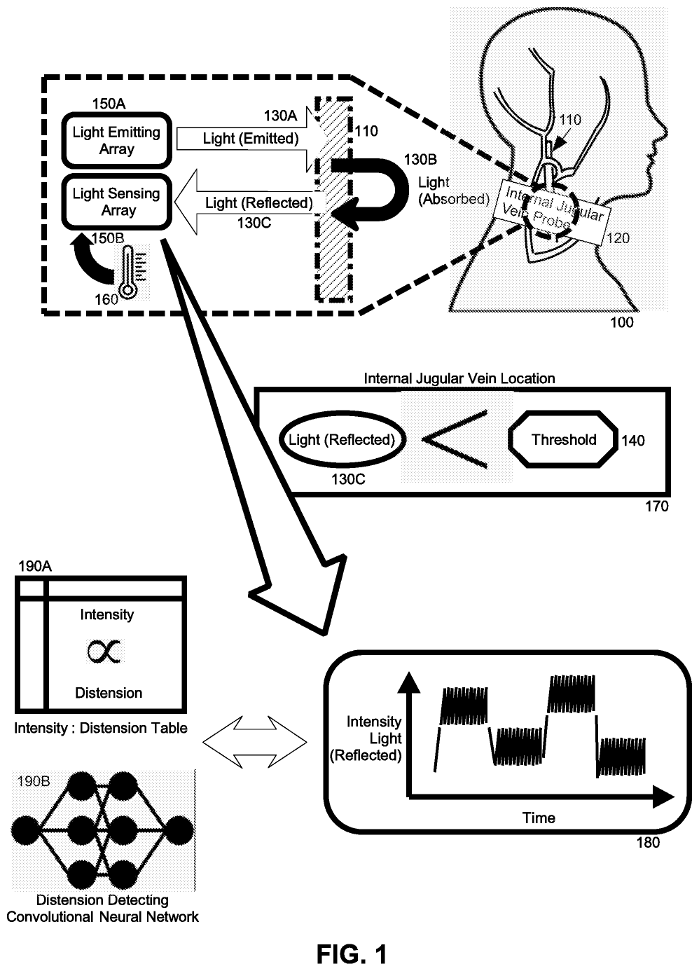

In further illustration, FIG. 1 pictorially shows a process for optically detecting a JV. As shown in FIG. 1 an JV probe 120 is placed on the surface of the neck 100 of a subject and in proximity to the JV 110 of the neck 100, for instance the IJV or the external JV. The JV probe 120 includes both a light emitting array 150A and a light sensing array 150B. The light emitting array 150A emits infrared light 130A of a wavelength greater than 700 nm directed at the JV 110. The light sensing array 150B senses reflected portions 130C of the emitted light 130A while absorbed portions 130B of the emitted light 130A are absorbed by blood in the JV 110. Optionally, a temperature sensor 160 monitors temperature changes at the light sensing array 150B and adjusts the values produced the sensors of the array 150B so as to account for sensor drift.

Of note, when the reflected portions 130C of the emitted light 130A is measured to fall below a threshold value 140 indicative of a peak amount of the absorbed portions 130B, it is determined that the JV has been located. Thereafter, a data set that includes a sequence of measurements over time of the reflected portions 130C of the emitted light 130A is displayed in display 180. As well, the sequence of measurements is then correlated to a distension value of the JV 110 by reference to a light intensity to distension table 190A. Alternatively, the sequence of measurements may be submitted to a convolutional neural network 190B trained to produce a probability of a particular distension value for the JV 110 based upon the sequence of measurements.

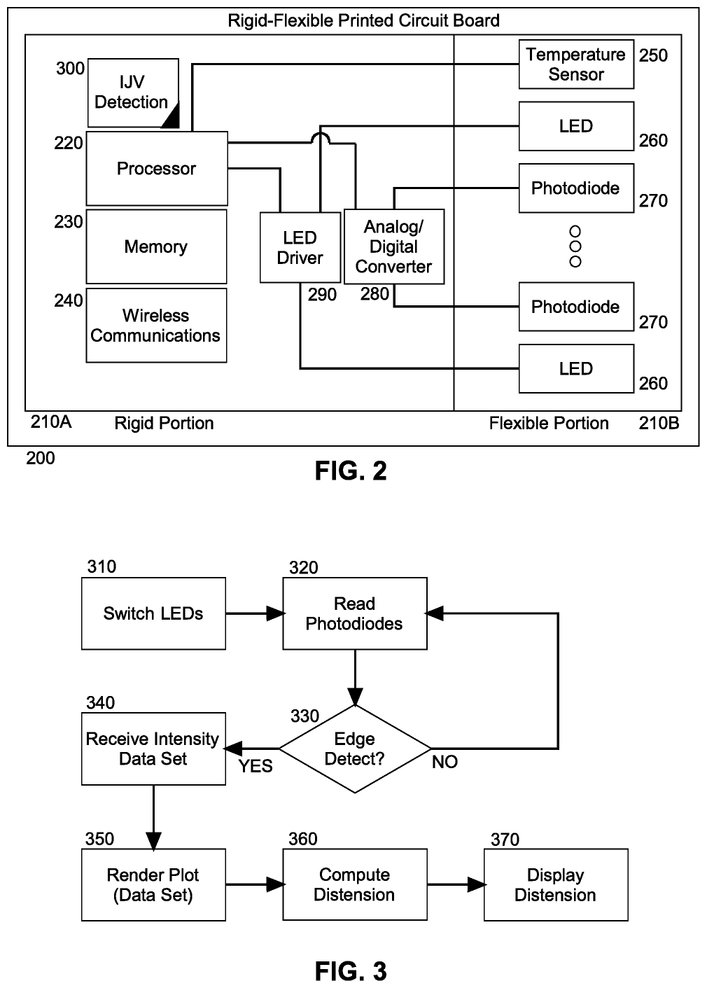

The process described in connection with FIG. 1 may be implemented within a data processing system. In more particular illustration, FIG. 2 schematically shows a computer data processing system adapted for JV optical detection. The system includes a rigid-flexible printed circuit board 200 that includes a rigid portion 210A and a flexible portion 210B. The flexible portion 210B includes each of a temperature sensor 250, an array of light emitting diodes (LEDs) 260 and an array of photodiodes 270 arranged in a grid pattern with the LEDs 260 dispersed therethrough. For instance, each of the LEDs 260 may be positioned at a corner of a rectangular grid of the photodiodes 270. Each of the LEDs 260 emits light at a wavelength that exceeds 700 nm. Alternatively, each of the LEDs 260 may be positioned evenly throughout the grid of photodiodes 270, with the LEDs 260 including an alternating arrangement of LEDs 260 emitting light at 740 nm and 850 nm.

The rigid portion 210A, in turn, has affixed thereto, a processor 220, memory 230 and wireless communications circuitry 240, for instance circuitry enabled to transmit wireless communications to a remote computing device such as a smartphone, tablet computer, smartwatch or personal computer. An LED driver 290 also is affixed to the rigid portion 210 and is adapted to switch on and off different ones of the LEDs 260. Finally, an analog to digital converter 280 is affixed to the rigid portion 210 and is adapted to generate a digital value for each analog signal received from a corresponding one of the photodiodes 270. Advantageously, the rigid-flexible printed circuit board 200 can be secured to a fabric strap such that the strap can secure the printed circuit board 200 to the neck of a subject with the flexible portion 210B bending around the curvature of then neck proximate to the JV of the neck, while the rigid portion 210 is positioned away from the JV near the back of the neck.

Notably, a JV detection module 300 executes by the processor 220. The JV detection module 300 includes a set of computer program instructions that, during execution by the processor 220, directs the LED driver 290 to switch on the LEDs 260 and to read from the analog to digital converter 280, digital values of intensity of the reflected portions of the light emitted by the LEDs 260 and received in the photodiodes 270. The program instructions are further enabled to compare the digital values to a threshold value tuned to indicate an expected peak absorption of the emitted light by blood in an JV. As such, to the extent that the digital values cross below the threshold value, the program instructions are enabled to determine that the JV has been located.

Once the JV has been located, the program instructions are additionally enabled to direct the LED driver 290 to switch the LEDs 260 on to emit additional infrared light targeting the location of the JV. In response, the program instructions read a set of intensity values from the analog to digital converter 280 and direct the processor to display a plot of the values indicative of distension of the JV. Optionally, the program instructions are enabled to direct the wireless communications circuitry 240 to transmit the set of intensity values to a remotely coupled display such as that within a smartphone, tablet computing device, smartwatch or personal computer.

In even yet further illustration of the operation of the JV detection module 300, FIG. 3 is a flow chart illustrating a process for optically detecting a JV in an JV probe. Beginning in block 310, the LEDs switch on and in block 320, values produced by the photodiodes are read into memory of the JV probe. In decision block 330, it is determined whether or not the JV is present and located, for instance the IJV. In this regard, the values produced by the photodiodes are compared to a threshold value indicative of a peak absorption of blood in the JV so as to determine when the JV is present in alignment with the LEDs. If so, in block 340, a set of intensity data values is received from the photodiodes and in block 350, the values are rendered in a display as a data plot. Then, in block 360, a distension of the JV is computed as being proportionate to the values and the computed distension is displayed in a display coupled to the JV probe.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

I claim:

1. A method for optically detecting a jugular vein (JV), the method comprising:
switching power in an JV probe to an array of light emitters emitting infrared light above seven-hundred nanometers (700 nm) onto a target area of a neck;
measuring in the JV probe an intensity of reflected portions of the infrared light; and,
locating the JV on the neck responsive to the measured intensity falling below a threshold value.

2. The method of claim 1, wherein the infrared light has a wavelength ranging from seven-hundred forty nanometers (740 nm) to eight-hundred fifty (850 nm).

3. The method of claim 1, further comprising:
collecting by the JV probe over a period of time, different values of the measured intensity of the reflected portions of the infrared light at the location of the JV of the neck; and,
displaying the different values in a time-based graph in a display coupled to the JV probe.

4. The method of claim 3, further comprising:
correlating the measured intensity at the location for each of the different values with corresponding degrees of distension; and,
displaying the corresponding degrees of distension in the display coupled to the JV probe.

5. A jugular vein (JV) optical detection system comprising:
an JV probe comprising a rigid-flexible printed circuit board substrate, a host computer comprising a processor, memory, and wireless communications circuitry, each affixed to a rigid portion of the substrate, and an array of light emitters emitting infrared light above seven-hundred nanometers (700 nm) and a corresponding array of infrared light sensors, both affixed to a flexible portion of the substrate; and,
an JV detection module comprising computer program instructions that when executing in the memory of the JV probe are enabled to perform:
switching power to the array of light emitters onto a target area of a neck;
measuring an intensity of reflected portions of the infrared light; and,
locating the JV on the neck responsive to the measured intensity falling below a threshold value.

6. The system of claim 5, wherein the infrared light has a wavelength ranging from seven-hundred forty nanometers (740 nm) to eight-hundred fifty (850 nm).

7. The system of claim 5, wherein the program instructions further collect over a period of time, different values of the measured intensity of the reflected portions of the infrared light at the location of the JV of the neck, and transmit the different values through the wireless communications circuitry to a remote computing device adapted to display the different values in a time-based graph.

8. The system of claim 7, wherein the program instructions further correlate the measured intensity at the location for each of the different values with corresponding degrees of distension and transmit the corresponding degrees of distension through the wireless communications circuitry to the remote computing device for inclusion in the display.

9. The system of claim 5, further comprising a temperature sensor affixed to the flexible portion of the substrate, wherein the program instructions further adjust the measured intensity of the reflected portions to account for sensor drift resulting from sensed temperature changes.

10. The system of claim 5, wherein the array of infrared light sensors comprises a multiplicity of photodiodes arranged in a grid pattern with staggered 850 nm and 740 nm light emitting diodes dispersed evenly throughout the grid of the photodiodes

11. The system of claim 8, further comprising a convolutional neural network trained to correlate different values for measured intensities at a location of an JV with different distension values, the program instructions submitting each of the different values of measured intensity to the convolutional neural network and receiving in response a probability that the different values correlate to respectively different degrees of distension.

12. A computer program product for optically detecting a jugular vein (JV), the computer program product including a non-transitory computer readable storage medium having program instructions embodied therewith, the program instructions executable by a device to cause the device to perform a method including:
switching power in an JV probe to an array of light emitters emitting infrared light above seven-hundred nanometers (700 nm) onto a target area of a neck;
measuring in the JV probe an intensity of reflected portions of the infrared light; and,
locating the JV on the neck responsive to the measured intensity falling below a threshold value.

13. The computer program product of claim 12, wherein the infrared light has a wavelength ranging from seven-hundred forty nanometers (740 nm) to eight-hundred fifty (850 nm).

14. The computer program product of claim 13, wherein the method further includes:
collecting by the JV probe over a period of time, different values of the measured intensity of the reflected portions of the infrared light at the location of the JV of the neck; and,
displaying the different values in a time-based graph in a display coupled to the JV probe.

15. The computer program product of claim 14, wherein the method further includes:
correlating the measured intensity at the location for each of the different values with corresponding degrees of distension; and,
displaying the corresponding degrees of distension in the display coupled to the JV probe.
